(19) United States
(12) Patent Application Publication
(10) Pub. No.: US 2013/0303762 A1
(43) Pub. Date: Nov. 14, 2013

(54) PROCESS FOR PREPARING BOSENTAN

(71) Applicants: Jayaraman Venkat Raman, Vadodara (IN); Samir Patel, Vadodara (IN); Samir Mistry, Vadodara (IN); Bhupendra Parmar, Vadodara (IN); Mukesh Timbadiya, Vadodara (IN); Malde Madam, Vadodara (IN)

(72) Inventors: Jayaraman Venkat Raman, Vadodara (IN); Samir Patel, Vadodara (IN); Samir Mistry, Vadodara (IN); Bhupendra Parmar, Vadodara (IN); Mukesh Timbadiya, Vadodara (IN); Malde Madam, Vadodara (IN)

(21) Appl. No.: 13/988,860

(22) PCT Filed: Nov. 10, 2011
(86) PCT No.: PCT/IB11/55010

§ 371 Date: Jul. 30, 2013

(30) Foreign Application Priority Data
Dec. 3, 2010 (IN) 3293/MUM/2010

(51) Int. Cl. C07D 239/52

(52) U.S. Cl. CPC C07D 239/52
USPC 544/296

(57) ABSTRACT

The present invention relates to an improved process for preparing Bosentan of formula (I).

### FIELD OF THE INVENTION

The present invention relates to an improved process for preparing Bosentan of formula (I).

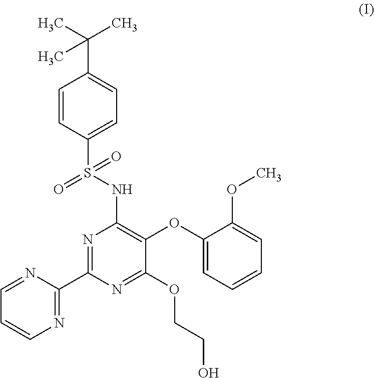 [SMILES: COc1ccccc1Oc1c(NS(=O)(=O)c2ccc(C(C)(C)C)cc2)nc(-c2ncccn2)nc1OCCO]

### BACKGROUND OF THE INVENTION

The chemical name of Bosentan is p-tert-butyl-N-[6-(2-hydroxyethoxy)-5-(o-methoxy phenoxy)-2-(2-pyrimidinyl)-4 pyrimidinyl]benzene sulfonamide. The current pharmaceutical product containing this drug is being sold by Actelion using the tradename Tracleer®, in the form of tablets.

Bosentan is used as Antihypertensive. It is Endothelin ETA Receptor Antagonists. It is also Endothelin ETB Receptor Antagonists. Tracleer® is indicated for the treatment of pulmonary arterial hypertension (WHO Group I) in patients with WHO Class II-IV symptoms, to improve exercise ability and decrease the rate of clinical worsening. It is also used in the treatment of cardiovascular disease, skin ulcer.

U.S. Pat. No. 5,292,740 describes a process for the preparation of Bosentan which is shown in the scheme-I.

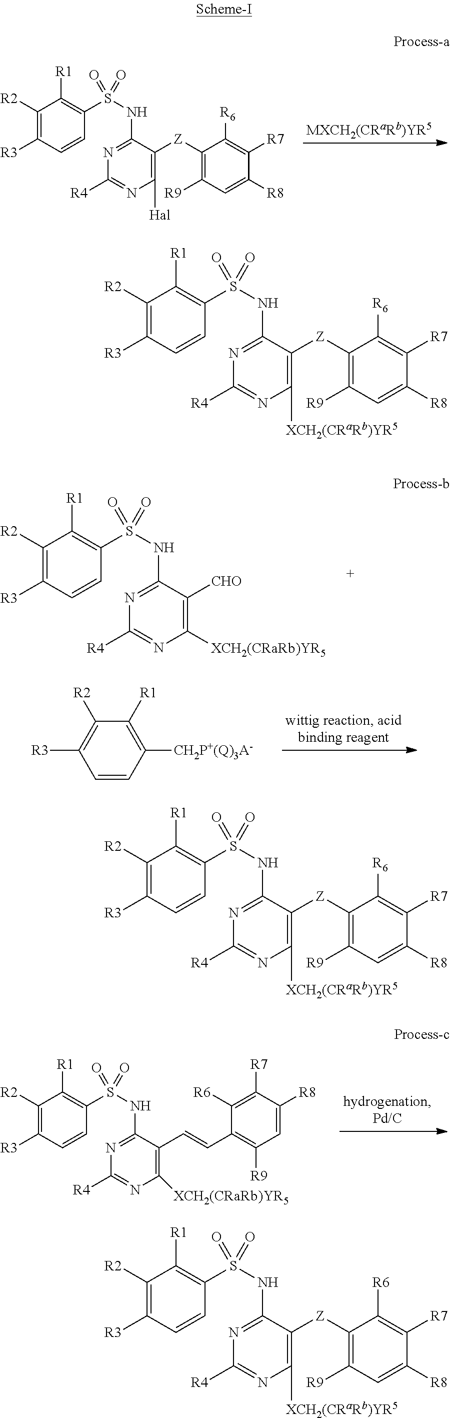

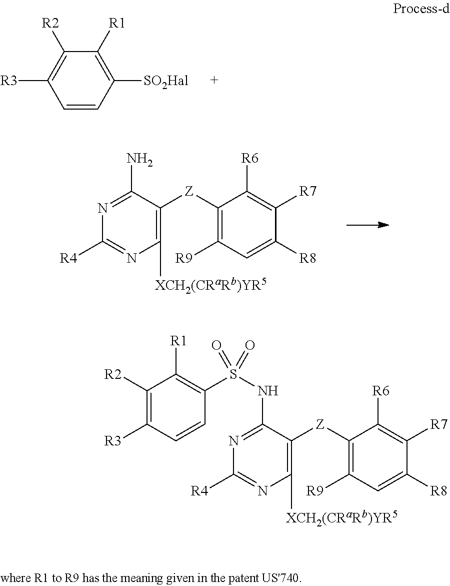

The patent discloses four various process routes from a to d. Process of route a involves reacting monohalide intermediate with glycol or its salt form or protected glycol to give Bosentan or its protected derivative. Process of route b involves reacting aldehyde intermediate with other intermediate via Wittig reaction. Process of route c involves reducing double bond present between two active moieties. Process of route d involves reaction between sulfonyl halide intermediate with amine intermediate.

The above process is not normally preferred at industrial scale as it requires chromatographic purification.

In the above process dimer impurity and deshydroxyethyl impurity is formed during process steps. The crude product needs to be purified by chromatography. It is very difficult to remove both these impurities from the final product. The impurities are having following structural formula.

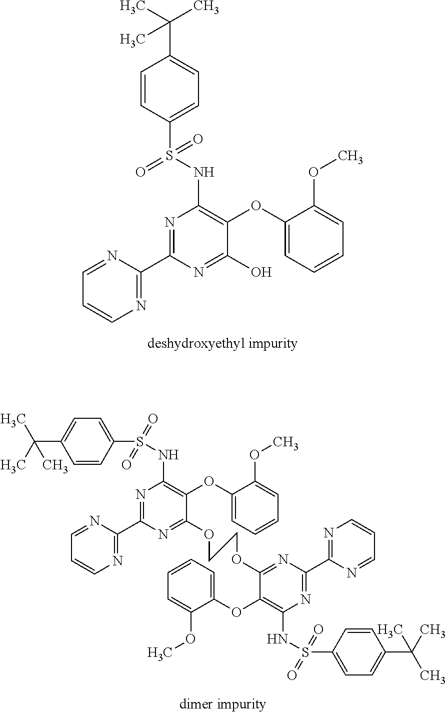

This dimer impurity is difficult to remove by conventional purification methods. According to ICH guideline, the drug should not have impurity more than the specified limit. For that it is required to limit the impurities present in the final drug compound.

It is therefore, a need to develop a smooth, industrially feasible process for preparing Bosentan wherein this both the impurity is controlled and removed from the final product. The present invention addresses these needs.

Present inventors have directed their research work towards developing a process for the preparation of Bosentan which is devoid of the above disadvantages. The present inventors developed an improved process for preparation of Bosentan via salt formation of Bosentan wherein purification of salt removes the unwanted impurity and finally gives highly pure Bosentan.

### SUMMARY OF THE INVENTION

Accordingly, it is an object of the present invention to provide an improved process for the preparation of Bosentan.

Another object of the present invention is to provide a process which gives Bosentan with high purity.

Yet another object of the present invention is to purify Bosentan potassium salt.

Another object of the present invention is to provide a process which is operationally simple and cost effective.

In another aspect of the present invention provides an improved process for preparation of Bosentan (I)

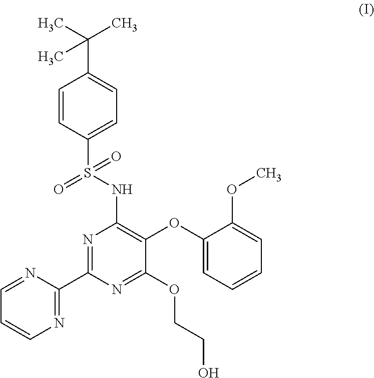 [SMILES: COc1ccccc1Oc1c(NS(=O)(=O)c2ccc(C(C)(C)C)cc2)nc(-c2ncccn2)nc1OCCO]

comprising a steps of reacting 4-tert butyl-N-[6-chloro-5-(2-methoxyphenoxy)-2-(pyrimidin-2-yl)pyrimidine-4-yl) benzene sulfonamide (IV) with ethylene glycol in the presence of sodium tert butoxide and potassium carbonate to give crude Bosentan potassium (V);

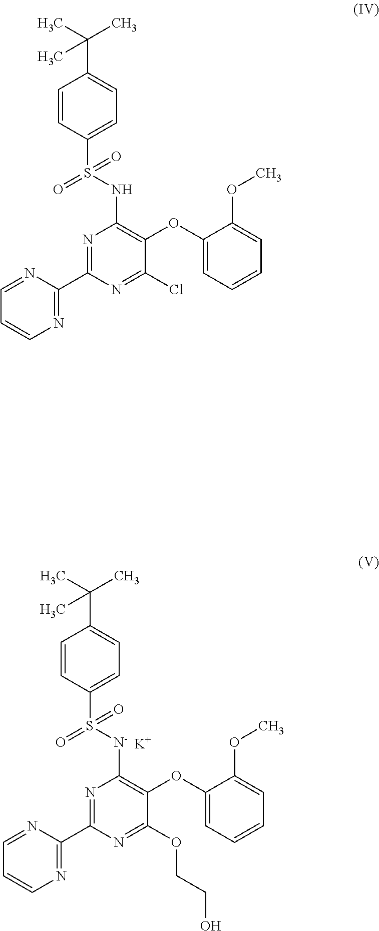 [SMILES: COc1ccccc1Oc1c(Cl)nc(-c2ncccn2)nc1NS(=O)(=O)c1ccc(C(C)(C)C)cc1 ; COc1ccccc1Oc1c([N-]S(=O)(=O)c2ccc(C(C)(C)C)cc2)nc(-c2ncccn2)nc1OCCO ; [K+]]

In another aspect of the present invention provides an improved process for preparation of Bosentan (I), comprising a steps of purifying crude Bosentan potassium (V) using dichloromethane.

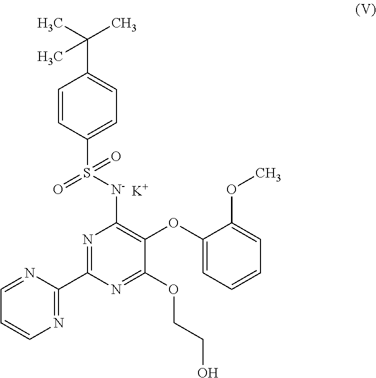 [SMILES: COc1ccccc1Oc1c([N-]S(=O)(=O)c2ccc(C(C)(C)C)cc2)nc(-c2ncccn2)nc1OCCO ; [K+]]

In another aspect of the present invention provides an improved process for preparation of Bosentan (I), comprising purification of compound of formula IV using ethyl acetate.

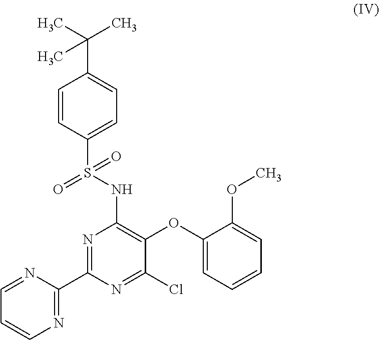 [SMILES: COc1ccccc1Oc1c(Cl)nc(-c2ncccn2)nc1NS(=O)(=O)c1ccc(C(C)(C)C)cc1]

Yet in another aspect of the present invention provides an improved process for preparation of Bosentan (I) comprising steps of

1). reacting dihalide compound of the formula (II) with 4-tert-butyl-benzene sulfonamide in presence of potassium carbonate in DMSO to obtain 4-tert butyl-N-[6-chloro-5-(2-methoxyphenoxy)-2-(pyrimidin-2-yl)pyrimidine-4-yl) benzenesulfonamide (IV),

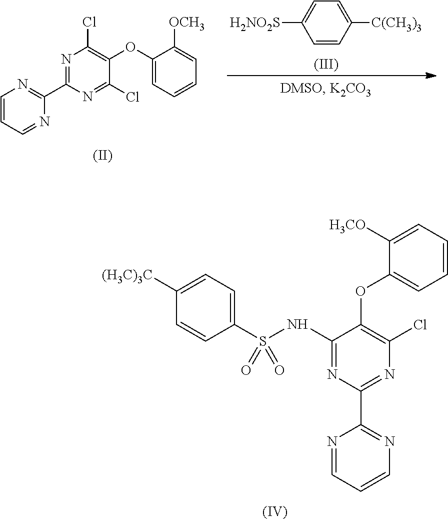

2). purifying compound of formula IV using ethyl acetate,

3). converting the compound of formula IV in to bosentan potassium (V) by treating with sodium tert butoxide and ethylene glycol and finally with potassium carbonate,

4). purifying bosentan potassium by using dichloro methane,

5). converting bosentan potassium in to bosentan by using Isopropyl alcohol, water and dil HCl,

6). treating wet crude bosentan with ethyl acetate,

7). Recrystallising crude bosentan with ethanol and water.

Accordingly, present invention provides Bosentan having impurity content less than 1.0%.

### BRIEF DESCRIPTION OF THE DRAWINGS

FIGS. 1 to 5 illustrates the different X-ray diffraction pattern of Bosentan potassium salt.

### DETAILED DESCRIPTION OF THE INVENTION:

The synthetic reaction scheme of the present invention is shown in the scheme-III.

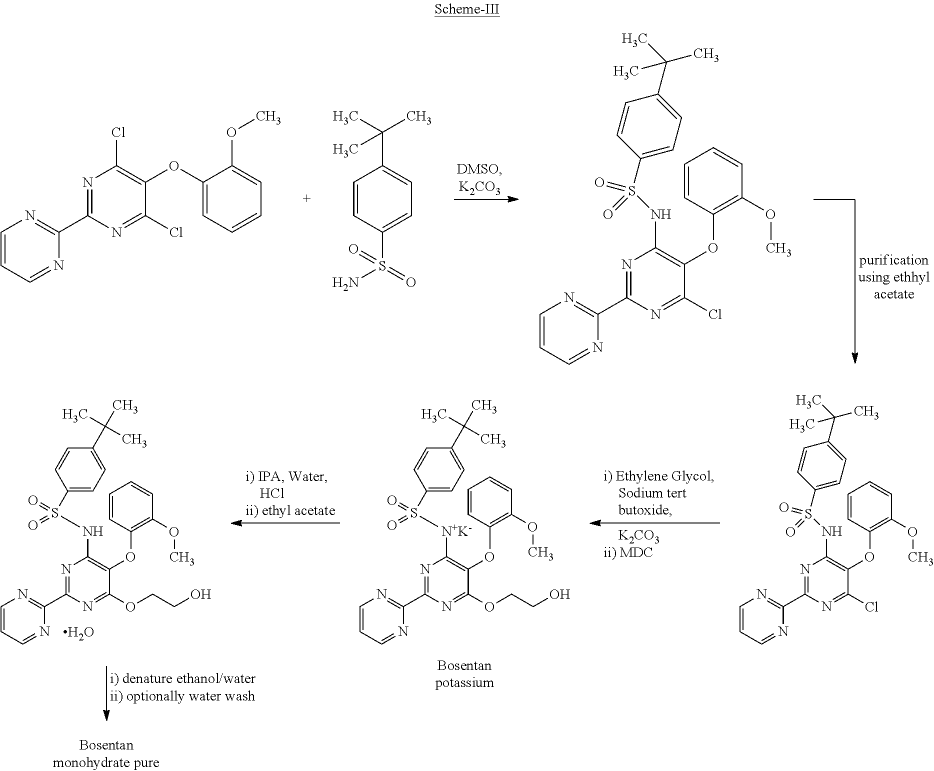

In the process of present invention, 4,6-dichloro-5-(2-methylphenoxy)-2,2′-bipyrimidine(II) is reacted with 4-tert-butylbenzene sulfonamide (III) in the presence of base and organic solvent to give 4-tert butyl-N-[6-chloro-5-(2-methoxyphenoxy)-2-(pyrimidin-2-yl)pyrimidine-4-yl) benzenesulfonamide (IV).

Base is selected from the group comprising alkali or alkaline earth metal hydroxide, carbonate, bicarbonate. The base is selected from NaOH, KOH, LiOH, NaHCO₃, KHCO₃, LiHCO₃, Na₂CO₃, K₂CO₃, Li₂CO₃, Mg(OH)₂, Ca(OH)₂, CaCO₃, MgCO₃, Ba(OH)₂, Be(OH)₂, BaCO₃, SrCO₃ and the like or mixtures thereof. The preferred base is K₂CO₃.

The examples of solvent are selected from a group comprising polar aprotic solvents such as dimethyl sulfoxide (DMSO), dimethyl formamide (DMF), dimethyl acetamide (DMAc) and the like or mixtures thereof. The preferred solvent is dimethyl sulfoxide (DMSO).

The reaction mixture is heated at 95 to 100° C. for about 4 to 5 hours till the completion of the reaction on thin layer chromatography (TLC). The reaction mixture is cooled, diluted with water and filtered to give wet solid compound which is again stirred with dilute hydrochloric acid at ambient temperature about half an hour. The solid is filtered, washed with DM Water and dried at 70 to 75° C. under vacuum for 8 to 10 hours. The solid thus obtained is purified by trituration from ethyl acetate. The volume of ethyl acetate is taken 7 to 9 times to the quantity of crude. The solid is added to ethyl acetate and heated at 75 to 80° C. for about 1 hour and then cooled at 30 to 40° C. and filtered. The wet caked is washed with ethyl acetate and dried in oven at 70 to 75° C. for 8 to 10 hours to give pure 4-tert butyl-N-[6-chloro-5-(2-methoxyphenoxy)-2-(pyrimidin-2-yl)pyrimidine-4-yl) benzenesulfonamide (IV)

In second step, the product obtained above is reacted with ethylene glycol in the presence of base such as alkali metal alkoxide. Potassium carbonate is added to give Bosentan potassium (V). The base can be selected from sodium tert-butoxide, potassium tert-butoxide, sodium methoxide, sodium ethoxide, potassium methoxide, potassium ethoxide, sodium propoxide and the like or mixtures thereof. The preferred base is sodium tert-butoxide. 4-tert butyl-N-[6-chloro-5-(2-methoxyphenoxy)-2-(pyrimidin-2-yl)pyrimidine-4-yl) benzenesul-fonamide (IV) is added to a mixture of ethylene glycol and sodium tert-butoxide and heated to a temperature of about 95° to about 100° C. for 7 to 8 hours till completion of the reaction by HPLC. Potassium carbonate is added to the reaction mixture at about 95° to about 100° C. and stirred for 30 min. DM Water is added to the reaction mixture and heated at 70 to 75° C. The reaction mixture was cooled at ambient temperature and the solid obtained is filtered, washed with water and suck dried to give wet crude Bosentan potassium (V). The deshydroxyethyl impurity is substantially reduced in this reaction step.

The wet crude Bosentan potassium (V) obtained in above step is purified to remove the unwanted impurities. The dimer impurity is substantially reduced in this purification step. The crude Bosentan potassium is added to dichloromethane and heated to reflux for about 30 min under stirring. Then cooled at RT. The solid obtained is filtered, washed with dichloromethane, suck dried and then dried in vacuum at 55° C. for about 8 to 10 hours to give pure Bosentan potassium salt.

The pure solid Bosentan potassium, water miscible solvent and DM Water are stirred at ambient temperature. The water miscible solvents are selected from alcohol, ketone, acetonitrile, DMF, DMSO, DMAc and the like. The preferred solvent is Isopropyl alcohol. Dilute acid solution with water is added to the reaction mixture dropwise. The acid is selected from the group of hydrochloric acid, hydrobromic acid, hydroiodic acid, sulfuric acid, nitric acid, acetic acid, formic acid and the like or mixtures thereof. 50% hydrochloric acid is preferred acid. 50% hydrochloric acid is added to it dropwise and stirred for about 2 to 3 hours at ambient temperature. The solid is filtered and washed with DM Water, suck dried and then litched with ethyl acetate. Again filtered it and dried in oven under vacuum to give Bosentan. Bosentan thus obtained can be purified by crystallization process. The crude solid is added to denatured ethanol and heated to reflux till clear solution is obtained. The reaction mixture was filtered and water is added in the filterate to obtain the product. Reaction mixture was stirred at ambient temperature for about 20 hours. The precipitates was filtered, washed with water and then dried in oven under vacuum at 55° C. for about 10 hours to give pure Bosentan.

The major advantage of this process is that the deshydroxyethyl impurity formation is very less in the second step compare to prior art process. The purification of Bosentan potassium reduces dimer impurity substantially. Combination of these two steps results in the minimization of unwanted impurities in the final Bosentan. Using prior art process, unwanted impurity is formed in more than 10% in Bosentan, whereas in present invention unwanted impurity is formed only 1%. The advantage of the present invention can be understand from the following data depicted in Table-1

TABLE 1

| | Bosentan prepared by | Bosentan prepared by |
|---|---|---|
| | Prior art process (before | Present invention |
| | column chromatography) | (before purification) |
| HPLC purity | 89.19% | 99.20% |
| % of dimer impurity | 0.47% | 0.31% |
| % of deshydroxyethyl | 8.99% | 0.15% |
| impurity | | |
| Yield (w/w) | 75-80% | 90-95% |

The process of the present invention has following advantages:

1. Deshydroxyethyl impurity is less formed.

2. Dimer impurity is removed by purification.

3. Easy isolation process.

4. Comparative quality and good yield is obtained.

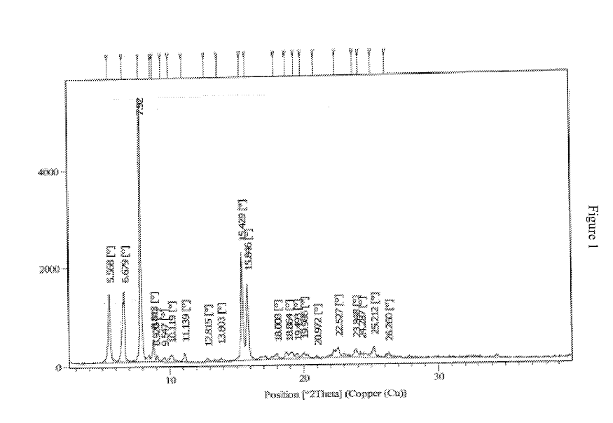

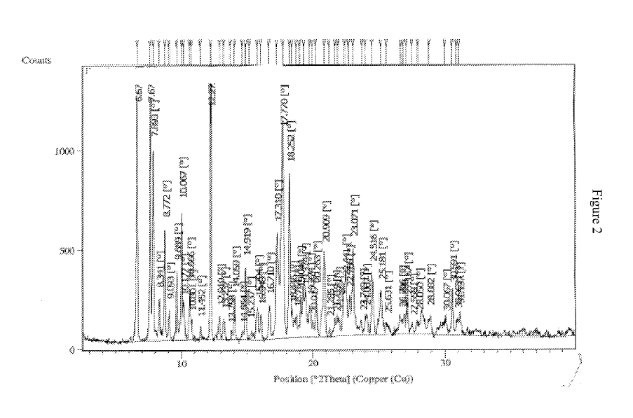

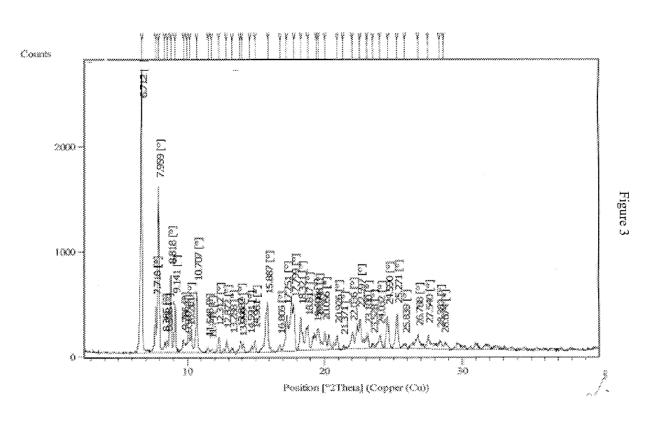

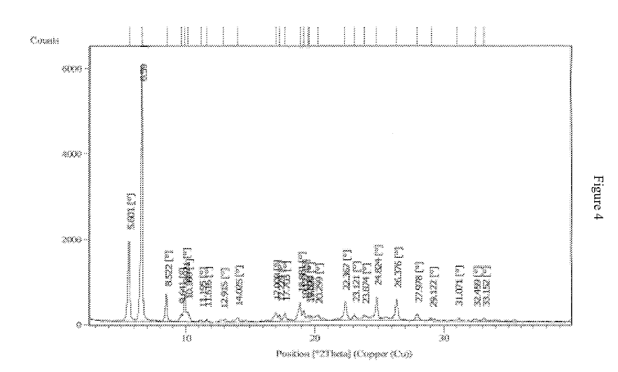

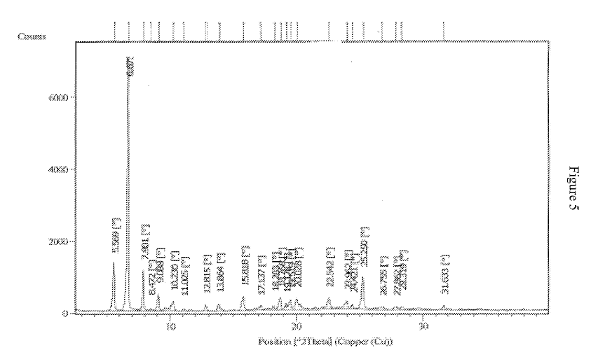

When the present inventors perform the preparation of Bosentan Potassium salt from the corresponding sodium salt it has been observed that the resulting compound can be of different crystalline nature. FIGS. 1 to 5 depicts the varieties of polymorphs of potassium salt of Bosentan, which are formed.

The following examples illustrate the invention further. It should be understood, however, that the invention is not confined to the specific limitations set forth in the individual examples but rather to the scope of the appended claims. cl EXAMPLE 1

### Preparation of 4-tert butyl-N-[6-chloro-5-(2-methoxyphenoxy)-2-(pyrimidin-2-yl) pyrimidine-4-yl) benzene sulfonamide (IV)

A mixture of 4,6-dichloro-5-(2-methylphenoxy)-2,2′- bipyrimidine(II) (100.0 g) is reacted with 4-tert-butylbenzene sulfonamide (III) (61.0 g) in the presence of Potassium carbonate (118.7 g) in dimethyl sulfoxide (300 ml) was heated at 95° to 100° C. for about 4 hours till the completion of the reaction. The reaction mixture is cooled at 30° to 40° C., diluted with water (800 ml) and stirred for 30 min. The reaction mixture was filtered and washed twice with water (100 ml×2). To this wet solid compound, dilute hydrochloric acid (50 ml in 500 ml water) (50.0 ml) is added at 25° to 35° C. and stirred for 1.0 hour. The solid compound was filtered, washed with water (100 ml×3) and dried at 70 to 75° C. under vacuum for 8 to 10 hours to give crude 4-tert butyl-N-[6-chloro-5-(2-methoxyphenoxy)-2-(pyrimidin-2-yl)pyrimidine-4-yl) benzenesulfonamide (IV) (145.0 g)

Purity by HPLC: 98.0%

Yield: 96.0%

### EXAMPLE 2

### Purification of 4-tert butyl-N-[6-chloro-5-(2-methoxyphenoxy)-2-(pyrimidin-2-yl) pyrimidine-4-yl) benzene sulfonamide (IV)

The crude solid 4-tert butyl-N-[6-chloro-5-(2-methoxyphenoxy)-2-(pyrimidin-2-yl)pyrimidine-4-yl) benzenesul-fonamide (IV) (100.0 g) was added to ethyl acetate (700.0 ml) and heated at 75 to 80° C. for about 1 hour and then cooled at 25 to 35° C. The reaction mixture was filtered and the wet cake was washed with ethyl acetate (100.0 ml). The wet solid was dried at 70° to 75° C. in oven under vacuum for 8 to 10 hours to give pure 4-tert butyl-N-[6-chloro-5-(2-methoxyphenoxy)-2-(pyrimidin-2-yl)pyrimidine-4-yl) benzene sulfonamide (IV) (90.0 g)

Purity by HPLC: 99.5%

Yield: 90.0%

### EXAMPLE 3

### Preparation of Bosentan potassium (V)

4-tert butyl-N-[6-chloro-5-(2-methoxyphenoxy)-2-(pyrimidin-2-yl) pyrimidine-4-yl) benzene sulfonamide (IV) (100.0 g) was added to a mixture of ethylene glycol (1000 ml) and sodium tert-butoxide (91.0 g) and heated at 90° C. for 3 hours till completion of the reaction by HPLC. Potassium carbonate (31.0 g) was added to the reaction mixture at 90° C. and stirred for 30 min. Water (1500 ml) was added to the reaction mixture and heated at 70° to 75° C. The reaction mixture was cooled at 25° to 35° C. and filtered. The solid was washed with water (100 ml×2), suck dried to give wet crude Bosentan potassium (V). The crude Bosentan potassium was added to dichloromethane (800 ml) and heated to reflux for about 30 min under stirring. Cooled the reaction mixture at 25° to 35° C. and stirred at the same temperature for 20 hours. The reaction mixture was filtered and solid obtained was washed with dichloromethane (100 ml×2). The solid was dried under vacuum at 55° C. to give pure Bosentan potassium salt (90.0 g)

Purity by HPLC: 99.0%

Yield: 90.0%

### EXAMPLE 4

### Preparation of Bosentan (I)

The pure solid Bosentan potassium (100.0 g), Isopropyl alcohol (500.0 ml) and DM

Water (500.0 ml) was stirred at 25° to 30° C. for 10 min. 50% hydrochloric acid (20.0 ml) was added dropwise to the reaction mixture and stirred for 3.0 hours at ambient temperature. The reaction mixture was filtered and washed with DM Water (100 ml×2), suck dried it. Then wet cake was refluxed with ethyl acetate (500 ml) for half an hour. The reaction mixture was cooled at 25° to 30° C. for 4 hours to give Bosentan (90.0 g). The reaction mixture was filtered and solid obtained was washed with ethyl acetate (100 ml). The solid was dried under vacuum at 55° C. to give Bosentan (85.0 g)

Purity by HPLC: 99.2%

Yield: 90.0%

### EXAMPLE 5

### Purification of Bosentan (I)

The crude Bosentan (100.0 g) was added to denatured ethanol (500 ml) and heated to reflux (70-80° C.) till clear reaction mixture was obtained. The reaction mixture was cooled at 40 to 45° C. and filtered through cartridge filter. DM water (500 ml) was added and reaction mixture was stirred for 6 hours at room temperature. Filtered the solid and washed with water and dried under vacuum at 55° C. for 8 to 10 hours to give pure Bosentan (70.0 g).

Purity by HPLC: 99.7%

Yield: 90.0%

1. A method for preparation of Bosentan (I) 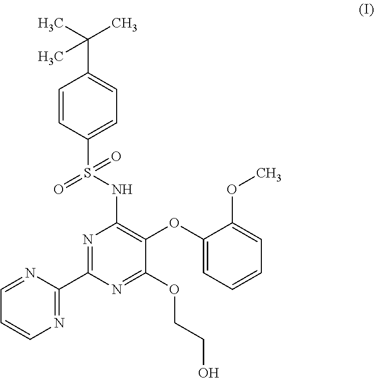 [SMILES: COc1ccccc1Oc1c(NS(=O)(=O)c2ccc(C(C)(C)C)cc2)nc(-c2ncccn2)nc1OCCO]
comprising a step of reacting 4-tert butyl-N-[6-chloro-5-(2-methoxyphenoxy)-2-(pyrimidin-2-yl)pyrimidine-4-yl) benzenesulfonamide (IV), 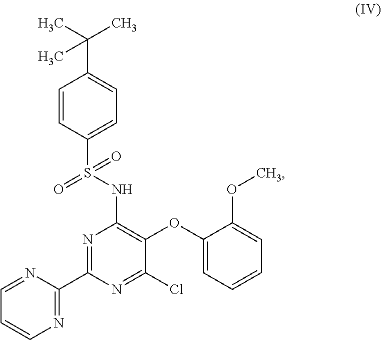 [SMILES: COc1ccccc1Oc1c(Cl)nc(-c2ncccn2)nc1NS(=O)(=O)c1ccc(C(C)(C)C)cc1]
with ethylene glycol in the presence of sodium tert butoxide and potassium carbonate to give crude Bosentan potassium (V),

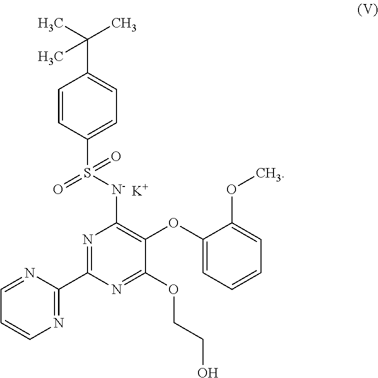 [SMILES: COc1ccccc1Oc1c([N-]S(=O)(=O)c2ccc(C(C)(C)C)cc2)nc(-c2ncccn2)nc1OCCO ; [K+]]

2. A method for preparation of Bosentan (I), comprising a step of purifying crude Bosentan potassium (V), 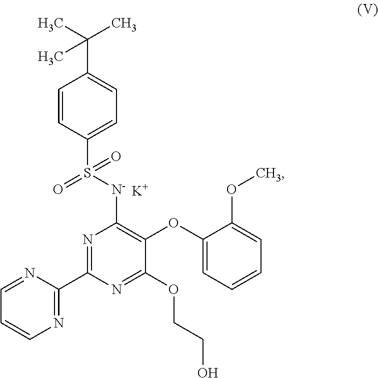 [SMILES: COc1ccccc1Oc1c([N-]S(=O)(=O)c2ccc(C(C)(C)C)cc2)nc(-c2ncccn2)nc1OCCO ; [K+]]
using dichloromethane.

3. A method for preparation of Bosentan (I), comprising purification of compound of formula IV, 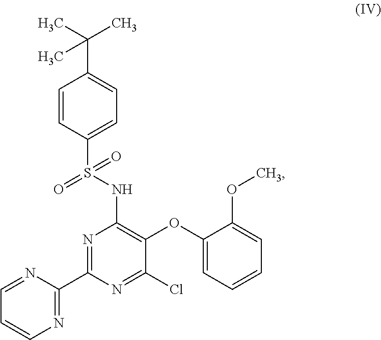 [SMILES: COc1ccccc1Oc1c(Cl)nc(-c2ncccn2)nc1NS(=O)(=O)c1ccc(C(C)(C)C)cc1]
using ethyl acetate.

4. A method for preparation of Bosentan (I),comprising steps of:
reacting dihalide compound of the formula (II), 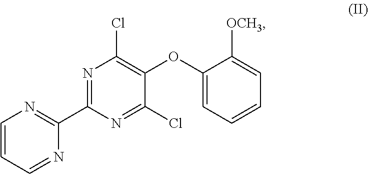 [SMILES: COc1ccccc1Oc1c(Cl)nc(-c2ncccn2)nc1Cl]
with 4-tert-butyl-benzene sulfonamide in presence of potassium carbonate in DMSO to obtain 4-tert butyl-N-[6-chloro-5-(2-methoxyphenoxy)-2-(pyrimidin-2-yl)pyrimidine-4-yl) benzenesulfonamide (IV), 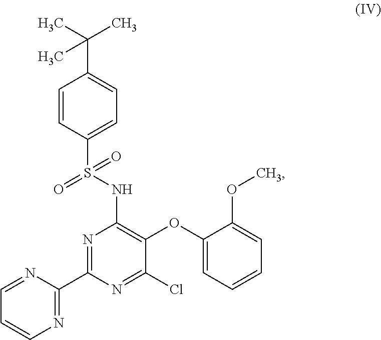 [SMILES: COc1ccccc1Oc1c(Cl)nc(-c2ncccn2)nc1NS(=O)(=O)c1ccc(C(C)(C)C)cc1]
(ii) purifying the compound of formula IV using ethyl acetate,
(iii) converting the compound of formula IV into bosentan potassium (V) by treating with sodium tert butoxide, ethylene glycol, and potassium carbonate,
(iv) purifying Bosentan potassium by using dichloromethane,
(v) converting Bosentan potassium into wet crude bosentan by using Isopropyl alcohol, water and dilute HCl,
(vi) treating the wet crude Bosentan with ethyl acetate,
(vii) recrystallising crude Bosentan with ethanol and water.
